(19) United States
(12) Patent Application Publication
(10) Pub. No.: US 2010/0168531 A1
(43) Pub. Date: Jul. 1, 2010

(54) RAPIDLY DEPLOYABLE SENSOR DESIGN FOR ENHANCED NONINVASIVE VITAL SIGN MONITORING

(75) Inventors: Phillip Andrew Shaltis, Sharon, MA (US); Andrew Tomas Reisner, Newtonville, MA (US)

Correspondence Address: Phillip Shaltis, 80 Cedar Street, Sharon, MA, 02067, US

(73) Assignees: Dr. Phillip Andrew Shaltis, Sharon, MA (US); Dr. Andrew Tomas Reisner, Newton, MA (US)

(21) Appl. No.: 12/604,043

(22) Filed: Oct. 22, 2009

Related U.S. Application Data
(60) Provisional application No. 61/107,429, filed on Oct. 22, 2008.

(51) Int. Cl. A61B 5/02; A61B 5/00

(52) U.S. Cl. 600/301; 600/500

(57) ABSTRACT

A new clip-type ring design for a rapidly-deployable triage sensor is described. The triage sensor is capable of measuring one or more parameters related to a patient's current health state. The device consists of two contoured halves which are designed to wrap around a finger like a ring. At least one of the halves is at least spring-loaded or motorized and is capable of opening or closing to allow for quick attachment to a wide range of finger shapes and sizes. The spring-loaded halves serve as both a means of securing the device to the patient as well as make it possible to measure patient health parameters such as systolic blood pressure, that are standard inputs to conventional triage methodologies. As data are acquired, the ring is able to transmit pertinent information wirelessly to medical responders for evaluation and decision making purposes.

### CROSS-REFERENCE TO RELATED APPLICATIONS

This application claims the benefit of PPA Ser. No. 61/107,429, filed Oct. 22, 2008 by the present inventors, which is incorporated by reference.

### FEDERALLY SPONSORED RESEARCH

Not Applicable

### SEQUENCE LISTING OR PROGRAM

Not Applicable

### BACKGROUND

1. Field of Invention

This invention pertains to a device for monitoring the health status of a patient and, more specifically to a mechanism for rapidly applying such a monitoring device to a wearer's extremity, and for improving and stabilizing subsequent monitoring device measurements from external disturbances.

2. Prior Art

Subsequent to a mass casualty incident (MCI), when there are more patients than can be instantaneously cared for, it is important to triage the patients. Triage is the utilitarian process of putting the patients into an order based on priority, so that available medical resources are allocated in as sensible a fashion as possible; proverbially, to do “the greatest good for the greatest number”. Most triage systems make use of blood pressure measurements, specifically, systolic blood pressure. For instance, the Revised Trauma Score, the Pre-hospital Index, the Triage Index, and the CRAMS scale (Circulation, Respiration, Abdomen, Motor, Speech) all involve systolic blood pressure data in a formula that yields a “severity score” for the patient, which quantifies the severity of the patient's condition after a traumatic event, as suggested, for example, by Kennedy, et al., Triage: techniques and applications in decision making, 28(2), Ann Emerg Med, (1996), pp. 136-44.

Also, while triage is important as an initial response to a mass casualty event, it is also essential that patients be continually re-assessed, because the rapid initial triage is imperfect at identification of major problems, and therefore, it is essential that those patients who, initially, do not appear to have severe injuries are monitored and re-evaluated as best as possible, though in practice, first responders will have many competing demands for their attention. It would be ideal to have the means to re-perform triage screening and scoring continually, through time, to identify patients with life-threatening conditions that were not initially appreciated.

One challenge of triage is that there is simply no suitable solution for measurement of SBP for situations such as those subsequent to an MCI. The familiar oscillometric blood pressure (BP) cuff is problematic to use in uncontrolled environments. U.S. Pat. No. 7,014,611 to Geddes et al. (2006) shows a noninvasive oscillometric blood pressure monitor. The familiar BP cuff must be wrapped around a patient's arm, and in many cases, neatly wrapped around itself so that it can be held in place by Velcro or Velcro-like fasteners. For the proper use of a conventional BP cuff, it is also important that the cuff is wrapped so that its inflatable bladder overlies the patient's brachial artery. If the cuff is wrapped with any physical irregularity, the BP cuff may not be well fastened by the Velcro, or the BP cuff may make erroneous measurements during inflation. Properly wrapping the cuff may take some care in normal clinical circumstances, and there is ample evidence that clinicians often do not use proper technique even for routine doctor visits, such as was reported by Jones, et al., Measuring blood pressure accurately: new and persistent challenges, 289(8), JAMA, (2003), pp. 1027-1030.

Considering that poor measurement technique involving BP cuffs is a challenge in a doctor's office appointment, it is undeniably a major challenge in the chaotic environment after an MCI, such as a train crash. In such circumstances it is very difficult to properly wrap the cuff, especially when patients may be uncooperative (due to panic, unconsciousness or severe pain), when patients may be wearing bulky clothing encumbering their upper arms, or when patients may be obese. As well, standard BP cuffs involve careful measurements of pulsations at given cuff pressures, and this technique is vulnerable to measurement errors caused by patient movement. Asking a patient to hold still in the chaotic aftermath of a MCI is also a challenge. What would be ideal is to have reliable, accurate blood pressure data, specifically SBP as well as other vital signs, available for mass casualty patients via a new tool or method that (a) could be quickly and easily applied by first responders without much effort; (b) require minimal patient cooperation; and (c) provide the means to continually reassess SBP with minimal risk of the device becoming dislodged or improperly positioned.

To date, no pre-existing device offers a solution to the problem of rapid reliable BP measurement for MCI triage and subsequent continual, reliable monitoring. The Vasotrac, described by U.S. Pat. No. 5,450,852 to Archibald, et al. (1995) is a blood pressure monitoring device that clamps around the wrist of a patient. The blood pressure measuring mechanism involves a small gelatinous bulb that is pressed to the skin overlying the radial artery, and then the device uses a basic variation of the well-known oscillometric technique to measure arterial blood pressure. The Vasotrac is correctly positioned by means of a wrist guard that fits against the the ulnar eminence, and thus aligns a small gelatinous bulb in a typical location of the radial artery. However, the device is prone to misalignment, because if the bulb is not overlying the radial artery, the measurement is erroneous. The device requires careful initial positioning and then delicate closing by an attentive, trained user, making it impractical to use in emergent care.

U.S. Pat. No. 5,490,523 to Isaacson, et al. (1996) shows a finger clip pulse oximeter. A pulse oximeter's fingertip probe uses a clamp-like design to allow for ease of placement on the end of a finger. However, this design is mechanically unstable in its attachment to the subject, because the device has a tendency to slip off the end of a naturally tapered fingertip in the setting of any forceful hand movement, as may be experienced in challenging environments. Moreover, in the fingertip, there is no means to measure arterial blood pressure, a previously noted critically important parameter for optimal MCI triage, since the blood vessels of the nailbed are arteriolar or smaller (not arterial).

The use of a ring-type device for the finger base has been described by the research group of the inventors. Two examples of such devices are U.S. Pat. No. 5,964,701 to Asada et al. (1999) and Shaltis, et al., Novel Design for a Wearable, Rapidly Deployable, Triage Sensor, Proceedings from the 27^{th} Annual International Conference of the IEEE-EMBS, (2006), pp 3567-3570. However, the former Asada ring does not have a mechanical design that is appropriate for deployment a chaotic MCI setting: placing a closed ring on individual subjects' fingers would be infeasible given the range of finger base sizes and knuckles blocking the rings' application, and possibility of minimally-cooperative patient because of pain, etc.

Regarding the latter ring-type device, described by Shaltis, et al., this prior art is a single-piece, horseshoe-shaped device. This single-piece design has two non-obvious limitations. First, the single-piece design is not able to accommodate a wide range of finger types and sizes. For example, a single-piece sensor will not firmly attach to both a full-grown adult and then to a small child. The second non-obvious limitation is that this design employs the well-known oscillometric method for blood pressure measurement. However, as noted above, oscillometry is suboptimal for MCI triage and continual monitoring because it is prone to measurement error unless the patients holds very still during the measurement, which is an unlikely human response after an MCI. An innovative mechanical design that is easy to attach on a wide range of patients, without careful placement, while enabling a more robust method of measuring blood pressure, would advance the prior art.

It has been previously noted that systolic blood pressure (SBP) can be determined by assaying for the loss of measureable pulsations distal to a cuff that is being inflated; the threshold cuff pressure that causes the loss of distal pulsations is close to SBP. Such implementations are taught, for example, by Talke, Measurement of systolic blood pressure using pulse oximetry during helicopter flight, 19, Crit Care Med, (1991), pp. 934-937, Talke, et al., Does measurement of systolic blood pressure with a pulse oximeter correlate with conventional methods?, 6, J Clin Monit, (1990), pp. 5-9, and McCluskey, et al., Out-of-hospital use of a pulse oximeter to determine systolic blood pressures, 11, Prehospital Disaster Med, (1996), pp. 105-107. However, these citations involve standard hospital instrumentation, i.e., a blood pressure cuff on the upper arm and a pulse oximetry probe on the finger. There is no mention that a compact, rapidly attached device that provides both controlled pressure application and pulsation measurement could be developed. Moreover, there is no suggestion that this technique, in conjunction with a compact, rapidly attached device, would enable rapid deployment of a blood pressure monitor in challenging, uncontrolled environments in which casualties' blood pressure must be assessed and continually re-measured.

### SUMMARY

{The Invention Summary will comport with the claims as filed.}

### DRAWINGS—FIGURES

In the drawings, closely related figures have the same number but different alphabetic suffixes.

FIG. 1 is an isometric view of the clip assembly constructed in accordance with the invention;

FIG. 2A is a front view of the clip assembly from the view of looking down the length of the patient's finger;

FIG. 2B is a front view of the clip assembly when opened prior to placement on the patient's finger;

FIG. 3 is a side view of the clip assembly showing the exposed sensor components and conditioning boards that are contained within the housing. This figure also shows the torsion spring used for elastically loading the clip;

FIG. 4 is a top view of the clip assembly showing exposed sensor components and a conditioning board; and

FIG. 5 is a personal point of view perspective looking down at the clip assembly as it is worn by a patient.

FIG. 6 is an isometric view of the lever with a removable attachment for resizing the structural clip of the sensor.

### DETAILED DESCRIPTION OF THE INVENTION

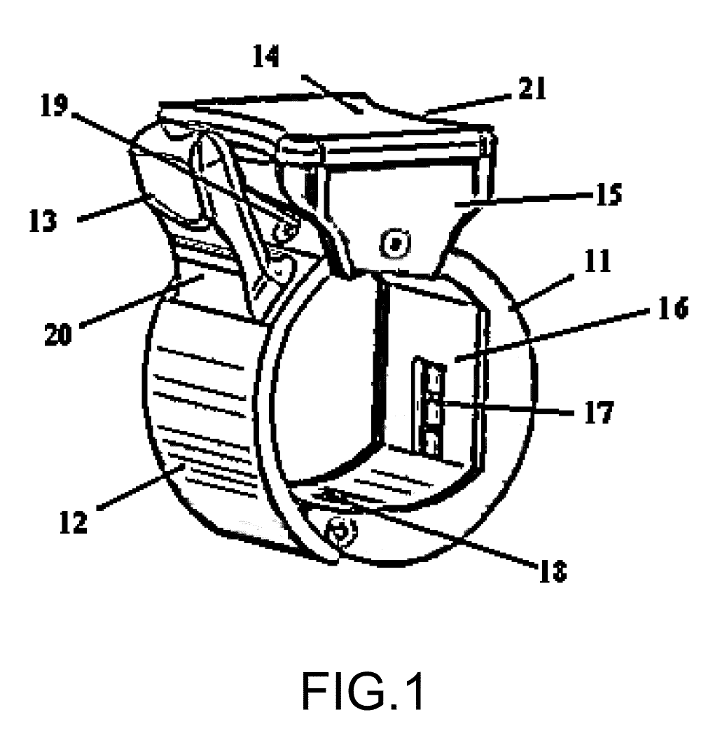

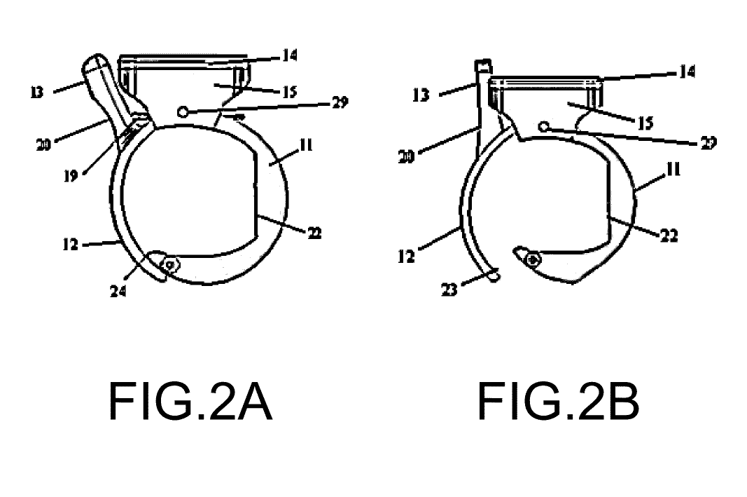

FIG. 1 is an isometric view of the clip assembly 11 taken from a view that is angled slightly relative to the length of a patient's finger in accordance with the invention. The top of the clip 14 covers a hollow top sensor housing 15 which contains signal conditioning electronics and a user interface. The structural clip 12 half of the assembly can be opened (FIG. 2B) by pinching together the top housing contour 21, which is fixed, and the lever 20 at the lever contour 13. The structural clip 12 rotates about a central guiding rod 29 and returns to a closed position, as pictured, due to a force applied by the arm of a torsion spring 19 on the lever 20. The stationary half of the clip assembly 11 is hollowed and contains both a detector array 17 and an emitter array 18 in this embodiment. The sensor arrays 17 and 18 and their associated electronics are shielded by an inner sensor cover 16. The inner sensor cover 16 also serves as a surface which presses directly against the finger of the patient. The clip assembly is made of a durable plastic such as ABS or could be made of an alternative durable material such as a light-weight metal. The sensors in this design are optical sensors but could consist of an alternative sensor modality capable of measuring either a volume or a pressure.

FIG. 2A is a front view of the ring in a closed configuration. In this configuration, the clip assembly 11 and the structural clip 12 are in contact with each other and form a closed ring. Note that a flat inner wall 22 would be positioned along the side of the finger and creates a uniform surface for performing measurements. Note also that the bottom of the clip assembly 11 contains a contoured end 24 which helps prevent pinching of the skin of the patient's finger when the ring closes.

FIG. 2B is a front view of the ring in a partially opened configuration. In this configuration, the lever 20 has been moved in centrally toward the top sensor housing 15. The inward movement of the lever 20 leads to a resized ring opening 23 at the bottom of the ring and provides for easy attachment to a wide range of finger sizes.

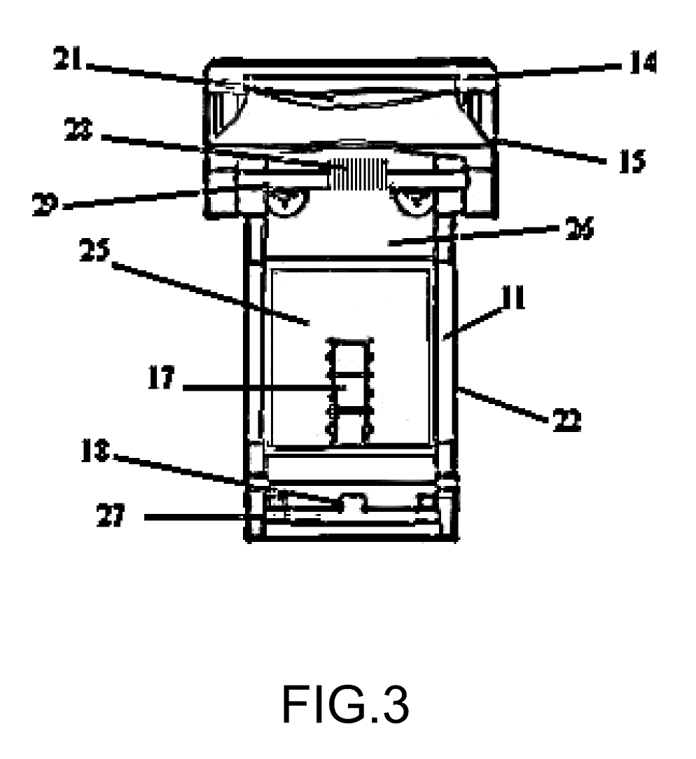

FIG. 3 is a side view of the ring with both the structural clip 12 and the inner sensor cover 16 removed. In this view we see the torsion spring 28 which applies a force to the lever (not shown) to keep the ring normally closed. The torsion spring 28 is kept in position by being placed about a guiding rod 29 running through the underside of the top sensor housing 15. The removed inner sensor cover 16 exposes the inside of the clip housing, making visible the detector array 17 and an accompanying detector conditioning board 25 positioned along the length of the flat inner wall 22. At the bottom of the ring the emitter array 18 and an accompanying emitter board 27 are also visible. In the present embodiment, the conditioning boards 25 and 27 are connected to a signal processing board (not shown) located within the top sensor housing 15.

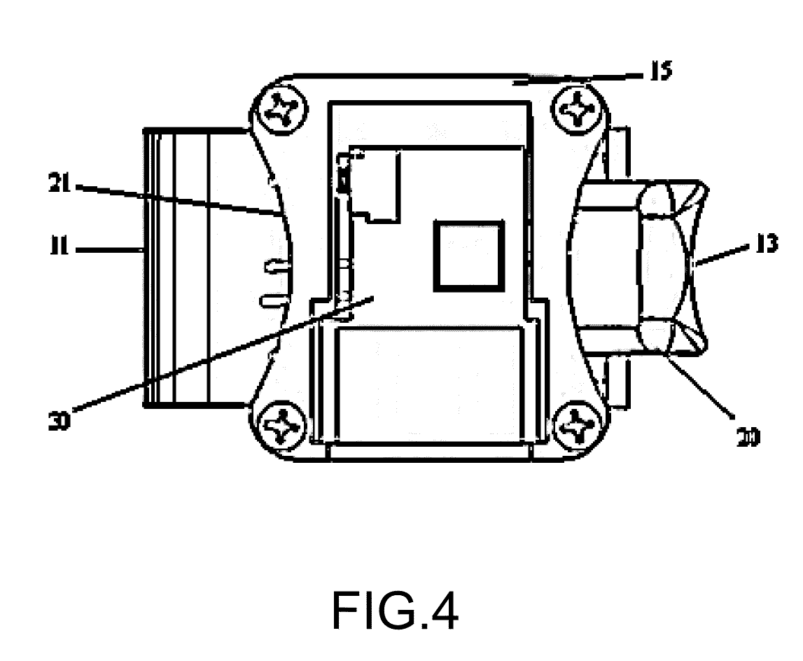

FIG. 4 is a top view of the ring with the clip top piece 14 removed. The view looks into the hollowed out portion of the top sensor housing 15 where a signal processing board 30 is situated. This board is connected to the conditioning boards (not shown) contained within the clip assembly 11. Note how the top housing contour 21 serves as a stationary surface for pinching on the clip assembly 11 side of the ring while a similar contoured surface on the opposite side of the top sensor housing 15 provides an open space where the lever 20 can fit when the ring is opened.

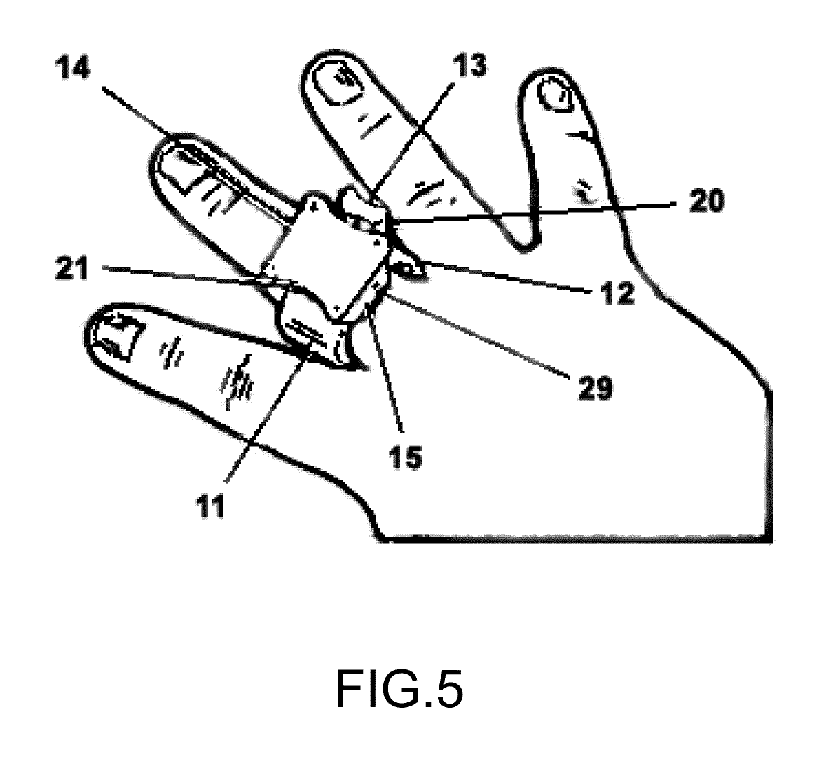

FIG. 5 is a personal point of view perspective looking down at the clip assembly as it is worn by a patient. Here, we clearly see the clip top piece 14 aligned along the length of the finger. The lever 20 and associated lever grip contour 13 are clearly visible on the ring finger side of the device. Note how these components are not in the way of the neighboring finger and would be easily accessible to a care provider during deployment. Opposite to the lever grip contour 13, we see the top housing contour 21. The top housing contour 21 serves as an additional location for a care provider to squeeze when opening the clip assembly. Within the top sensor housing 15, we see the guiding rod 29 around which both the torsion spring (not pictured) and the structural clip 12 pivot. Note how the structural clip 12 half of the assembly consists of a thin and smooth design to maximize patient comfort between the fingers. Opposite to the structural clip 12 is the clip assembly half of the ring. Again, this is the portion of the design that contains the detector array (not pictured) and the emitter array (not pictured).

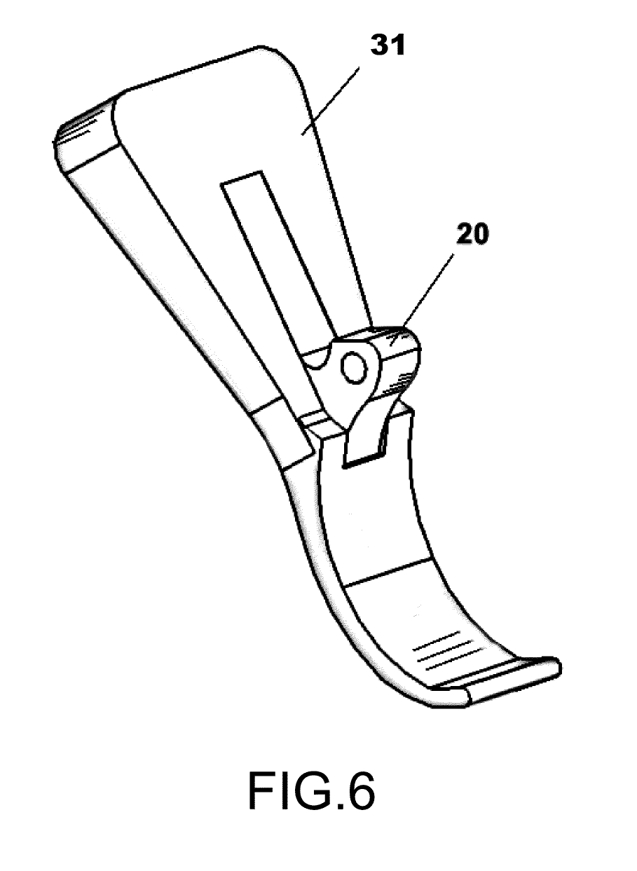

FIG. 6 is an isometric view of the lever with a removable attachment for resizing the structural clip of the sensor. The lever 20 is attached to a removable structural clip 31. The removable structural clip allows the curvature and shape of the sensor unit assembly to be changed to accommodate a wide range of patients when used in the field.

### Operation

In its preferred embodiment, the device is applied to the bare finger of a trauma casualty. The device displays a clear visual indicator, located in the clip top piece 14, that its battery charges are sufficient for prolonged field use, indicating to a medical responder which individual units are ready for field use. A medical responder pinches the levers 20 and 21 of the device (one lever 21 is actually the side of the top sensor housing 15), which opens the clip portions of the device 11 and 12, as shown in FIG. 2A and FIG. 2B. These levers 20 and 21 are contoured 13 and covered in a high-friction surface, to make them easy to grip by medical responders, even in demanding environments, e.g., rain. The height of the top sensor housing 15 is enough for the responders to pinch, but minimal enough that the top sensor housing 15 does not protrude in an obtrusive way when worn by a casualty. In an alternative embodiment, the clip portions of the device 11 and 12 each possess a joint, and when the medical responder pinches the levers 20 and 21, the clip portions 11 and 12 open, but also, there is articulation at each joint so that the distal elements of each clip flares open. In other words, the entire clip mechanism opens up due to rotation both at the guiding rod 29, but also due to rotation in the joints along each clip portion 11 and 12. When the medical responder releases the levers 20 and 21, the device closes with two points of rotation for each clip portion 11 and 12, and so the device firmly encloses the base of the subject's finger with reduced risk of pinching skin.

In the preferred embodiment, the device is placed around the thumb, pointer finger, or pinky finger of the casualty. The device has two contoured halves 11 and 12, matching the contour of a typical human finger. The bulkier of the two halves 11 contains all the sensor elements 17 and 18 and electronics 25 and 26. This bulkier half 11 is clearly labeled, to communicate to the responder that it should ideally face externally, facing open space and away from any fingers, so that it will be more comfortable to wear for the casualty. The structural half 12 is very thin, a structural component without any other functionality. Because it is so thin, it can comfortably be worn between two fingers, e.g., pinky and ring finger or pointer finger and long finger. When the medical responder releases the levers 20 and 21, the spring-loaded clips 19 close and the device holds securely about the base of the finger, as shown in FIG. 5. As the two halves close, they overlap in a tapered manner, and both have rounded edges 24, so that the device completely encircles the finger without pinching the skin of the casualty. In one alternative embodiment, the two halves do not overlap, which also avoids pinching the skin of the casualty.

In another alternative embodiment, the devices are held open by some mechanical means, but when the mechanism is tripped, i.e. a button is depressed, the restraint is removed and the spring-loaded jaws automatically close around a finger. In another alternative embodiment, there is a simple mechanism to activate the sensing electronics, such as a button inside the band of the ring that is depressed when the sensor fits onto a finger, so that its batteries are not consumed prior to deployment. In one alternative embodiment, there is a simple locking mechanism, such as a latch, so that, once the jaws close around a finger, the locking mechanism holds the jaws closed. In alternative embodiments, the locking mechanism can either be automatic or alternatively, set and unset by the medical responder. In alternative embodiments, the device may have the means to be re-sized, to fit on larger and smaller (e.g. pediatric) digits. For instance, there may be the means to change the angle of the jaws at the pivot. Alternatively, there may be the means to adjust the curvature of one or both of the sensor halves 11 and 12, or to replace one of the sensor halves using a removable structural clip, such as is illustrated in 31.

Once the sensor is fit to the finger of a casualty, the spring-loaded pivot 12, 19, 20 holds the sensor in place. The compliant material within the ring ensures a snug fit, and that the device remains comfortable, too. In an alternative embodiment, there are small grooves within the interior surface, which establishes channels for sweat and water to drain. The top sensor housing 15 and the lever 20 restrict the rotation of the ring around the finger, so that the sensor elements 17 and 18 cannot become grossly misaligned with the digital artery at the base of the finger. The emitter array 18 illuminates the base of the finger, and a detector array 17 records the reflected optical signal. The device automatically identifies the optimal photodetector for measuring the PPG, automatically optimizes the signal, and begins measuring the continuous PPG signal, from which heart rate, oxygen saturation, and respiratory rate are computed. The threshold pressure above which the pulsatile PPG signal is lost is taken as the systolic blood pressure (SBP), which is measured on a continual basis. In an alternative embodiment, SBP is taken as a function of this threshold pressure. This SBP functionality requires using information from a motion sensor, to ensure that SBP is measured only when the patient's hand is in a known, stable orientation, e.g., horizontal; and to account for SBP measurements when the hand's orientation changes, e.g. pointing down or pointing up, which can alter the SBP that is measured in the hand. This same position sensing functionality may be applied to other physiologic measurements.

The pressure necessary for occluding the pulsatile PPG signal is provided by at least one of the following, the spring loading of the device's hinge, elasticity in the structural components of the ring, or physical action by the responder. The torsion spring 28 may be joined with or even replaced by a small motor that can be used to automatically close the clip portions 11 and 12 of the ring to apply pressure to the trauma casualty's extremity.

The device measures one or more physiologic signals, and processes them within the top sensor housing 15. The device wirelessly transmits numeric vital signs every few seconds. The wireless transmissions from each deployed device may be received and monitored by a mobile computing unit, such as a phone or other portable computing device, or by a stationary base station. In alternative embodiments, the device may transmit full waveform data, or it may merely transmit a sparse summary priority status for triage purposes, e.g., “red”, “green”, “yellow”, or “black”, which is generated by automated processing of the physiologic data with a triage algorithm. In an alternative embodiment, the device emits a unique signal to help remote caregivers locate the individual casualty, who may be in need of urgent medical therapy. For instance, a remote medical responder may notice that the casualty condition has gone from “yellow” (urgent) to “red” (emergent), and may want to identify that casualty from amongst a large number of monitored casualties. Through wireless electronic communication, the medical responders may be able to initiate a homing signal that is either electromechanical or acoustic in nature. In an alternative embodiment, the acoustic speaker is also able to transmit verbal instructions to the casualty, sent from the medical responders by wireless electronic communication.

The device transmits data related to the status of the sensor, including a rating of the reliability of its physiologic measurements (e.g., if the waveform data appear physiologic or noisy) and related to its battery status. The device is able to automatically determine, and transmit, whether or not the device is applied to a finger. In the preferred embodiment, this is determined by a pressure sensor within the band of the ring 11. In an alternative embodiment, alternative sensor modalities are employed, including the inner-ring photodetectors, which can detect the presence or absence of ambient light, as well as thermocouples both on the inner-ring and the exterior housing. In the alternative embodiment, an algorithm utilizes all the available sensor data to determine when the ring is attached to a finger. In an alternative embodiment, the algorithm uses the presence or absence of inner-ring temperature only when the ambient temperature, measured by the exterior housing thermocouple, is well below physiologic ranges of human body temperature.

In an alternative embodiment, the device is networked to a monitoring station that is observed by medical responders. In the alternative embodiment, the sensor data are processed, and all the aforementioned data are displayed, specifically, any measurements made directly by the sensor; any indices related to the quality of the measurements; and lastly, any overall assessment of the casualty that results from automated data processing of a ring sensor's data, which may include, but is not limited to: severity color-coding (e.g., red, green, etc.); severity scoring (e.g., the revised triage score, or a novel severity score); numerical triage priorities; and specific casualty conditions (e.g., major hemorrhage).

In an alternative embodiment, the device is altered so that it is large enough to fit over the wrist or ankle or other location on the extremity of a casualty, while preserving all the other aforementioned functionality.

From the description above, a number of advantages of some embodiments of our rapidly deployable sensor design become evident:

(a) A care provider is able to use a simple, familiar, and relatively effortless pinching motion to rapidly attach the proposed device to a patient's extremity, minimizing the time required to begin assessment of a trauma casualty and establish the means to automatically monitor the casualty through time.

(b) The device completely encircles the circumference of the patient's extremity, so that it is securely and comfortably attached, while applying a suitably uniform loading about the instrumented segment of the extremity.

(c) Using a plurality of rigid components attached by one or more hinges provides a means to adjust the angle of the rigid components so that the device can conform to a wide range of finger shapes and sizes.

(d) Employing an unconventional method to measure SBP, rather than employing conventional Oscillometry, provides the means to assess SBP without the unrealistic expectation that supervised or unsupervised MCI casualties would be willing to remain voluntarily motionless during the time it takes to make an Oscillometric BP measurement. Together with heart rate and respiratory rate, SBP is an essential metric of circulatory function in trauma patients, and a standard input to a plurality of well-known triage methods and triage scoring systems (The photoplethysmographic sensors offer the means to measure and monitor HR and RR).

(e) The encircling design of the sensor will permit accurate patient monitoring in a wide range of device orientations, making it easier to deploy rapidly.

### CONCLUSION, RAMIFICATIONS, AND SCOPE

Accordingly the reader will see that the rapidly deployable sensors of the various embodiments can be attached quickly, securely, and comfortably, to obtain vital signs from a patient in emergent monitoring scenarios, and demonstrates a design that can provide robust measurements of vital signs including systolic blood pressure even in the aftermath of an MCI, where consistent cooperation of the casualties is unlikely. While the above description contains many specificities, these should not be construed as limitations on the scope of any embodiment, but as exemplifications of the presently preferred embodiments thereof. Many other ramifications and variations are possible within the teachings of the various embodiments. For example, the device may have other shapes, such as a round, square, or triangular top; the hinge mechanism may be made of a different compliant mechanism, such as a flexible polymer or have a bi-stable, uni-body design; a compliant material may be added to the inside surface of the device to provide additional comfort for the patient and shield the sensors from environmental disturbances, etc.

Thus the scope of the invention should be determined by the appended claims and their legal equivalents, and not by the examples given.

The invention claimed is:

1. A rapidly-deployable system for monitoring the health status of a patient, the monitoring system consisting of the following:
a plurality of rigid bodies that are pivotably attached by hinges and are disposed to surround a segment of a patient's extremity;
at least one protrusion that provides a mechanism for adjusting the size of the open space between the rigid bodies;
a sensing apparatus that is able to detect pulsations from a circulatory metric from a majority of orientations about the patient's extremity segment;
a mechanical means for applying a loading to the segment of extremity sandwiched between the rigid bodies;
a sensing apparatus for measuring the loading applied by the rigid bodies to the instrumented segment of extremity; and
a processor for conditioning the circulatory metrics and determining the loading above which there is the loss of pulsations in the instrumented segment of extremity.

2. A monitoring system according to claim 1, wherein there is a system for transmitting patient information to a remote processing station.

3. A monitoring system according to claim 2, wherein the transmission to a remote processing station is performed wirelessly.

4. A monitoring system according to claim 2, wherein there is at least one additional health status metric based on at least one of pulse rate, respiratory rate, skin temperature, extremity motion, arm height, and blood constituent concentration.

5. A monitoring system according to claim 2, wherein the instrumented segment of extremity is the base of a finger.

6. A monitoring system according to claim 2, wherein one sensor is a plethysmographic sensor consisting of light emitting and detecting elements.

7. A monitoring system according to claim 2, wherein the mechanical means to apply a loading to the segment of extremity sandwiched between the rigid bodies is a spring.

8. A monitoring system according to claim 2, wherein the mechanical means to apply a loading to the segment of extremity sandwiched between the rigid bodies is a small motor.

9. A monitoring system according to claim 2, in which blood pressure is determined to be a function of the loading above which there are no measurable pulsations in the instrumented segment of the extremity.

10. A monitoring system according to claim 9, wherein the blood pressure is systolic blood pressure.

11. A monitoring system according to claim 2, wherein at least one part of the monitoring system serves as a housing for electronics.

12. A monitoring system according to claim 1, wherein the inner surface may have smooth irregularities to prevent a build-up of sweat.

13. A monitoring system according to claim 1, wherein the inner surface may contain additional material for softening the monitoring system's contact with the wearer's skin.

14. A monitoring system according to claim 1, wherein the curvature of the rigid bodies can be mechanically adjusted to accommodate different diameters of extremity segments.

15. A monitoring system according to claim 2, wherein the system provides a visual indication as to whether or not the ring is attached.

16. A monitoring system as in claim 3, in which the sensor measurement is a load sensor within the interior of the ring.

17. A monitoring system as in claim 3, in which the sensor measurement is a temperature sensor.

18. A monitoring system as in claim 16, in which an algorithm uses a plurality of sensor measurements to identify the likelihood that the sensor is attached to a finger.

20. A monitoring system according to claim 2, wherein the sensor and software system provide the caregiver with a signal quality metric from improved vital sign monitoring.
